(19) United States
(12) Patent Application Publication
(10) Pub. No.: US 2007/0208173 A1
(43) Pub. Date: Sep. 6, 2007

(54) CRYSTALLINE HYDRATES OF CEFDINIR CALCIUM SALT

(76) Inventors: Ron Christopher Kelly, San Mateo, CA (US); Jeffrey Scott Stults, West Lafayette, IN (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/680,844

(22) Filed: Mar. 1, 2007

Related U.S. Application Data
(60) Provisional application No. 60/778,310, filed on Mar. 2, 2006.

(51) Int. Cl. C07D 501/14; C07D 501/00

(52) U.S. Cl. 540/222

(57) ABSTRACT

Crystalline hydrates of (6R-(6α,7β(Z))-7-((2-amino-4-thiazolyl)(hydroxyimino)acetyl)amino)-3-ethenyl-8-oxo-5-thia-1-azabicyclo[4.2.0]oct-2-ene-2-carboxylic acid (cefdinir), calcium salt, ways to make them, compositions comprising them and made with them, and methods of treatment using them are disclosed.

This application claims priority to U.S. Provisional Application Ser. No. 60/778,310, Mar. 2, 2006.

### FIELD OF THE INVENTION

This invention pertains to crystalline hydrates of (6R-(6α,7β(Z))-7-((2-amino-4-thiazolyl)(hydroxyimino)acetyl)amino)-3-ethenyl-8-oxo-5-thia-1-azabicyclo[4.2.0]oct-2-ene-2-carboxylic acid (cefdinir), calcium salt, ways to make them, compositions comprising them and made with them, and methods of treatment using them.

### BACKGROUND OF THE INVENTION

Cefdinir, marketed in the United States as OMNICEF®, is an antibiotic available as capsules or particles for suspension. Cefdinir is useful for treating infections resulting from bacteria such as Staphylococcus aureus, Streptococcus pneumoniae, Streptococcus pyogenes, Hemophilus influenzae, Moraxella catarrhalis, E. coli, Klebsiella and Proteus mirabilis.

Crystallinity of salts of compounds and solvates thereof may effect, among other physical and mechanical properties, their solubility, dissolution rate, hardness, compressibility and melting point. Because the ease of manufacture and use of cefdinir is dependent on its solubility, dissolution rate, hardness, compressibility and melting point, there is an existing need in the chemical and therapeutic arts for identification of novel crystalline forms of salts of cefdinir and hydrates thereof and ways of reproducibly making them.

### BRIEF DESCRIPTION OF THE FIGURES

FIG. 1 shows a powder X-ray diffraction (PXRD) pattern of cefdinir, calcium salt pentahydrate.

### SUMMARY OF THE INVENTION

One embodiment of this invention pertains to isolated crystalline hydrates of cefdinir, calcium salt.

Another embodiment pertains to crystalline cefdinir, calcium salt pentahydrate.

Still another embodiment pertains to crystalline cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

### DETAILED DESCRIPTION OF THE INVENTION

This invention pertains to discovery of novel crystalline hydrates of cefdinir, calcium salt, such as, for example, crystalline cefdinir, calcium salt pentahydrate, ways to make them having substantial crystalline, chemical and isomeric purity, ways to characterize them, compositions containing them and methods of treating bacterial infections using them.

One embodiment of this invention pertains to crystalline hydrates of cefdinir, calcium salt.

Another embodiment pertains to a crystalline cefdinir, calcium salt pentahydrate.

Still another embodiment pertains to crystalline cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

Still another embodiment pertains to crystalline cefdinir, calcium salt pentahydrate having substantial crystalline purity, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

Still another embodiment pertains to crystalline cefdinir, calcium salt pentahydrate having substantial crystalline purity, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

Still another embodiment pertains to crystalline cefdinir, calcium salt pentahydrate having substantial crystalline purity, substantial chemical purity and substantial isomeric purity, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

Still another embodiment pertains to compositions comprising or made with an excipient and a crystalline hydrate of cefdinir, calcium salt.

Still another embodiment pertains to compositions comprising or made with an excipient and cefdinir, calcium salt pentahydrate.

Still another embodiment pertains to compositions comprising or made with an excipient and cefdinir, calcium salt pentahydrate, said cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

Still another embodiment pertains to methods of treating bacterial infection in a mammal comprising administering thereto a therapeutically effective amount of a crystalline hydrate of cefdinir, calcium salt.

Still another embodiment pertains to methods of treating bacterial infection in a mammal comprising administering thereto a therapeutically effective amount of a crystalline cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

Still another embodiment pertains to mixtures comprising a crystalline hydrate of cefdinir, calcium salt and cefdinir.

Still another embodiment pertains to a process for making a crystalline cefdinir, calcium salt pentahydrate, said process comprising:

providing a mixture comprising cefdinir, water and acetone;

causing crystalline cefdinir, calcium salt pentahydrate to exist in said mixture, said crystalline cefdinir, calcium salt pentahydrate, when isolated and measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°; and

isolating said crystalline cefdinir, calcium salt pentahydrate.

Still another embodiment pertains to a process for making a crystalline cefdinir, calcium salt pentahydrate, said process comprising:

providing a mixture comprising cefdinir, water and acetone;

combining said mixture and calcium acetate and allowing said crystalline cefdinir, calcium salt pentahydrate to form, said crystalline cefdinir, calcium salt pentahydrate, when isolated and measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°; and

isolating said crystalline cefdinir, calcium salt pentahydrate.

Still another embodiment pertains to a process for making a crystalline cefdinir, calcium salt pentahydrate, said process comprising:

providing a mixture comprising cefdinir, water, acetone and calcium acetate and allowing said mixture to exist, with or without agitation, for about 7 days; and

isolating said crystalline cefdinir, calcium salt pentahydrate.

Still another embodiment pertains to a crystalline cefdinir, calcium salt pentahydrate prepared by the foregoing processes.

Still another embodiment pertains to a process for making Cefdinir Crystalline Form A, said process comprising:

providing a mixture comprising a hydrate of cefdinir, calcium salt, water and an alcohol;

causing crystalline Cefdinir Crystalline Form A to exist in said mixture, said crystalline Cefdinir Crystalline Form A, when isolated and measured with radiation at 1.54178 Å, comprising a powder diffraction pattern with at least five peaks having respective 2θ values of about 11.8°, 12.6°, 14.7°, 16.6°, 17.8°, 18.9°, 19.2°, 21.5°, 22.0°, 23.4°, 24.5°, 25.4°, 26.9°, 27.7°, 28.1° and 29.7°; and

isolating said Cefdinir Crystalline Form A.

Still another embodiment pertains to Cefdinir Crystalline Form A prepared by the foregoing process.

Still another embodiment pertains to a process for making Cefdinir Crystalline Form A, said process comprising:

providing a mixture comprising a hydrate of cefdinir, calcium salt, water and methanol between about 75° C. to about 100° C.;

adjusting the pH of the mixture to about 1 to about 4 and causing crystalline Cefdinir Crystalline Form A to form in said mixture, said crystalline Cefdinir Crystalline Form, when isolated and measured with radiation at 1.54178 Å, comprising a powder diffraction pattern with at least five peaks having respective 2θ values of about 11.8°, 12.6°, 14.7°, 16.6°, 17.8°, 18.9°, 19.2°, 21.5°, 22.0°, 23.4°, 24.5°, 25.4°, 26.9°, 27.7°, 28.1° and 29.7°; and

isolating said Cefdinir Crystalline Form A.

Still another embodiment pertains to Cefdinir Crystalline Form A prepared by the foregoing process.

Cefdinir, calcium salt may also referred to herein as calcium 7-(2-(2-aminothiazol-4-yl)-2-hydroxyminoacetamido)-3-vinyl-3-cephem-4-carboxylate (syn-isomer) and calcium (6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylate (syn-isomer).

The term “bacterial infections,” means infections resulting from Staphylococcus aureus, Streptococcus pneumoniae, Streptococcus pyogenes, Hemophilus influenzae, Moraxella catarrhalis, E. coli, Klebsiella, Proteus mirabilis or a combination thereof.

The term “cefdinir” as used herein, means amorphous cefdinir, crystalline cefdinir, a hydrate of cefdinir, cefdinir in solution, semisolid cefdinir and mixtures thereof.

The term “hydrate of cefdinir,” as used herein, means a cefdinir lower hydrate or an iso-structural hydrate thereof and crystalline cefdinir trihemihydrate with or without surface water.

The terms “crystalline,” as used herein, means having a regularly repeating arrangement of molecules which is maintained over a long range or external face planes.

The term “crystalline cefdinir,” as used herein, means a particular crystalline cefdinir, including, but not limited to, Cefdinir Crystalline Form A, when isolated and measured with radiation at 1.54178 Å, comprising a powder diffraction pattern with at least five peaks having respective 2θ values of about 11.8°, 12.6°, 14.7°, 16.6°, 17.8°, 18.9°, 19.2°, 21.5°, 22.0°, 23.4°, 24.5°, 25.4°, 26.9°, 27.7°, 28.1° and 29.7°.

The term “amorphous,” as used herein, means a supercooled liquid substance or a viscous liquid which appears solid and does not have a regularly repeating arrangement of molecules which is maintained over a long range. Amorphous substances do not have a melting point but soften or flow above their glass transition temperature.

The term “hydrate,” as used herein, means having water associated therewith.

The term “cefdinir lowerhydrate,” as used herein, means crystalline cefdinir•H₂O to crystalline cefdinir•1.5H₂O, all of which are characterized in the monoclinic crystal system and C2 space group when measured with radiation at 0.7107 Å, by respective lattice parameters a, b and c of 23.95 Å±0.03 Å, 4.984 Å±0.006 Å and 15.87 Å±0.01 Å and β of 108.88°±0.02°.

The term “iso-structural hydrate of a cefdinir lowerhydrate,” as used herein, means a crystalline hydrate of cefdinir•H₂O to cefdinir•1.5H₂O, all of which are characterized by substantially similar unit cell parameters and powder X-ray diffraction pattern of the corresponding lower hydrate but with a different water content in the unit cell, wherein the term “substantially similar,” as used herein, means falling within the range of the unit cell parameters.

The term “cefdinir trihemihydrate,” as used herein, means crystalline cefdinir•3.5H₂O characterized in the monoclinic crystal system and C2 space group, when measured with radiation at 0.7107 Å, by respective lattice parameters a, b and c of 23.95 Å±0.03 Å, 4.984 Å±0.006 Å and 15.87 Å±0.01 Å and β of 108.88°±0.02°.

Unless stated otherwise, percentages herein are weight/weight (w/w) percentages.

The term “substantial crystalline purity,” as used herein, means at least about 95% crystalline purity, preferably about 97% crystalline purity, more preferably about 99% crystalline purity, and most preferably about 100% crystalline purity.

The term “crystalline purity,” as used herein, means percentage of a particular crystalline form of a substance in a sample which may contain one or more than one other crystalline forms of the substance, or a mixture thereof.

The term “substantial chemical purity,” as used herein, means about 95% chemical purity, preferably about 97% chemical purity, more preferably about 98% chemical purity, and most preferably about 100% chemical purity.

The term “chemical purity,” as used herein, means percentage of a particular compound in a sample. For example, crystalline cefdinir, calcium salt hydrates, may contain varying amounts of impurities including, but not limited to, (2Z)-N-((5aR,6R)-3-methyl-1,7-dioxo-1,4,6,7-tetrahydro-3H,5aH-azeto[2,1-b]furo[3,4-d][1,3]thiazin-6-yl)-2-(2-amino-1,3-thiazol-4-yl)-2-(hydroxyimino)ethanamide, (2R)-(((2Z)-2-(2-amino-1,3-thiazol-4-yl)-2-(hydroxyimino)acetyl)amino)((2R)-5-methyl-7-oxo-1,2,5,7-tetrahydro-4H-furo[3,4-d][1,3]thiazin-2-yl)ethanoic acid, (2Z)-2-(2-amino-1,3-thiazol-4-yl)-2-(hydroxyimino)-N-(((2R)-5-methyl-7-oxo-1,2,5,7-tetrahydro-4H-furo[3,4-d][1,3]thiazin-2-yl)methyl)ethanamide, (2Z)-2-(2-amino-1,3-thiazol-4-yl)-N-(2,2-dihydroxyethyl)-2-(hydroxyimino)ethanamide, (2R,5Z)-2-[(R)-(((2Z)-2-(2-amino-1,3-thiazol-4-yl)-2-(hydroxyimino)ethanoacetyl)(carboxy)methyl)-5-ethylidene-5,6-dihydro-2H-1,3-thiazine-4-carboxylic acid, (2R,5Z)-2-((((2Z)-2-(2-amino-1,3-thiazol-4-yl)-2-(hydroxyimino)acetyl)amino)methyl)-5-ethylidene-5,6-dihydro-2H-1,3-thiazine-4-carboxylic acid, (((2Z)-2-(2-amino-1,3-thiazol-4-yl)-2-(hydroxyimino)acetyl)amino)acetic acid, (6R,7R)-7-(((2Z)-2-(2-amino-1,3-thiazol-4-yl)-2-(hydroxyimino)acetyl)amino)-3-methyl-8-oxo-5-thia-1-azabicyclo[4.2.0]oct-2-ene-2-carboxylic acid, (6R,7R)-7-(((2-amino-1,3-thiazol-4-yl)acetyl)amino)-8-oxo-3-vinyl-5-thia-1-azabicyclo[4.2.0]oct-2-ene-2-carboxylic acid, (6R,7R)-7-(((4-hydroxyisoxazol-3-yl)carbonyl)amino)-8-oxo-3-vinyl-5-thia-1-azabicyclo[4.2.0]oct-2-ene-2-carboxylic acid, (6R,7R)-7-(((2Z)-2-(2-amino-1,3-thiazol-4-yl)-2-(hydroxyimino)acetyl)amino)-8-oxo-3-vinyl-5-thia-1-azabicyclo[4.2.0]oct-2-ene-2-carboxylic acid 5-oxide, (6R,7R)-7-(((2-amino-1,3-thiazol-4-yl)(oxo)acetyl)amino)-8-oxo-3-vinyl-5-thia-1-azabicyclo[4.2.0]oct-2-ene-2-carboxylic acid, (2E)-2-(2-amino-1,3-thiazol-4-yl)-2-(hydroxyimino)-N-(((2R)-5-methyl-7-oxo-1,2,5,7-tetrahydro-4H-furo[3,4-d][1,3]thiazin-2-yl)methyl)ethanamide, mixtures thereof and the like.

Cefdinir, calcium salt hydrates may also have a degredant wherein the oxime moiety of cefdinir is in the anti-configuration.

The term “substantial isomeric purity,” as used herein, means cefdinir, calcium salt hydrates having an isomeric excess greater than about 95% cefdinir, syn-isomer, preferably greater than about 97% cefdinir, syn-isomer, more preferably greater than about 99% cefdinir, syn-isomer, and most preferably about 100% cefdinir, syn-isomer, wherein the remainder is the degredant of cefdinir having oxime moiety in the anti-configuration.

The term “isomeric excess,” as used herein, means amount of one isomer of a compound in a sample which may contain another isomer of the same compound.

The term “alcohol,” as used herein, means methanol, ethanol or a combination thereof.

It is meant to be understood that each component of a mixture comprising a hydrate of cefdinir, calcium salt and cefdinir may have varying degrees of chemical and isomeric purity and that, in a preferred embodiment for the practice of this invention, each component of the mixture is substantially chemically and isomerically pure.

The term “solvent,” as used herein, means a liquid in which a compound is soluble or partially soluble enough at a given concentration to dissolve or partially dissolve the compound. Examples of solvents for the practice of this invention include, but are not limited to, water and organic solvents including, but not limited to, methanol, ethanol, tetrahydrofuran, acetone, acetonitrile, N,N-dimethylformamide, dimethylsulfoxide, ethyl acetate, isopropyl acetate, dichloromethane, pyridine and the like.

The term “anti-solvent,” as used herein, means a liquid in which a compound is insoluble enough at a given concentration or temperature to not dissolve the compound or to precipitate the compound from solution.

Solvents and anti-solvents may be mixed with or without separation of phases.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present.

The term “acid,” as used herein, means a compound having at least one acidic proton. Examples of acids for the practice of this invention include, but are not limited to, hydrochloric acid, hydrobromic acid, trifluoroacetic acid, trichloroacetic acid, sulfuric acid, phosphoric acid and the like.

The term “base,” as used herein, means a compound capable of accepting a proton. Examples of bases for the practice of this invention include, but are not limited to, sodium carbonate, sodium bicarbonate, potassium carbonate, potassium bicarbonate, calcium acetate, triethylamine, diisopropylethylamine and the like.

The term “isolating” as used herein, means separating a crystalline hydrate of cefdinir, calcium salt and solvent, anti-solvent, or a mixture of solvent anti-solvent. This is typically accomplished by means such as centrifugation, filtration with or without vacuum, filtration with positive pressure, distillation, evaporation or a combination thereof.

A therapeutically acceptable amount of a crystalline hydrate of cefdinir, calcium salt depends on recipient of treatment, disorder being treated and severity thereof, composition containing it, time of administration, route of administration, duration of treatment, its potency, its rate of clearance and whether or not another drug is co-administered. The amount of a cefdinir used to make a composition to be administered daily to a patient in a single dose or in divided doses is from about 0.03 to about 200 mg/kg body weight. Single dose compositions contain these amounts or a combination of submultiples thereof.

A crystalline hydrate of cefdinir, calcium salt may be administered with or without an excipient. Excipients include but are not limited to, for example, encapsulating materials and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising or made with a crystalline hydrate of cefdinir, calcium salt to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising or made with a crystalline hydrate of cefdinir, calcium salt to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising or made with a crystalline hydrate of cefdinir, calcium salt to be administered osmotically include, for example, chlorofluorohydrocarbons, ethanol, water, mixtures thereof and the like. Excipients for preparation of compositions comprising or made with a crystalline hydrate of cefdinir, calcium salt to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising or made with a crystalline hydrate of cefdinir, calcium salt to be administered rectally or vaginally include, but are not limited to, cocoa butter, polyethylene glycol, wax, mixtures thereof and the like.

The following examples are presented to provide what is believed to be the most useful and readily understood description of procedures and conceptual aspects of this invention.

### EXAMPLE 1

### benzhydryl(6R-(6α,7β(Z)))-7-(((2-amino-1,3-thiazol-4-yl)-2-(hydroxyimino)acetyl)amino)-8-oxo-3-vinyl-5-thia-1-azabicyclo[4.2.0]oct-2-ene-2-carboxylate

A mixture of benzhydryl (6R,7R)-7-((4-bromo-3-oxobutanoyl)amino)-8-oxo-3-vinyl-5-thia-1-azabicyclo[4.2.0]oct-2-ene-2-carboxylate (10 g) in dichloromethane (70 mL) and acetic acid (25 mL) at −5° C. was treated with isoamylnitrite (3.5 mL), stirred for 40 minutes, treated with acetylacetone (4 g), stirred for 30 minutes, treated with thiourea (3 g), stirred for 3 hours, treated with ethyl acetate (70 mL) and diisopropyl ether (100 mL) and filtered. The filtrant was dried under vacuum.

### EXAMPLE 2

### (6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylic acid (cefdinir, syn-isomer)

A mixture of trifluroacetic acid and anisole at 5° C. was treated with EXAMPLE 1, stirred for 1 hour, added to diisopropyl ether (150 mL) and filtered. The filtrant was dissolved in THF (10 mL) and ethyl acetate (10 mL), and the mixture was extracted with aqueous sodium bicarbonate. The extract was washed with ethyl acetate while keeping the pH at 5, adjusted to pH 2.2 with 10% hydrochloric acid, stirred for 1 hour at 0° C. and filtered. The filtrant was dried under vacuum.

Alternatively, boron trifluoride etherate (5 mL) at 10° C. was treated with EXAMPLE 1 (5 g) in anisole (20 mL) and acetic acid (5 mL), stirred for 20 minutes, poured into 1:1:1 THF/ethyl acetate/water (300 mL), and adjusted to pH 6 with 20% aqueous sodium hydroxide. The water layer was isolated, adjusted to pH 6, if necessary, washed with ethyl acetate, and eluted through aluminum oxide with 3% aqueous sodium acetate. Elutes containing desired product were collected, adjusted to pH 4 with 10% hydrochloric acid and eluted through nonionic absorption resin Diaion HP-20® (Mitsubishi Chemical Industries) with 20% aqueous acetone. Elutes containing desired product were collected, concentrated, adjusted to pH 2 with 10% hydrochloric acid and filtered.

### EXAMPLE 3

### calcium (6R-(6α,7β(Z)))-7-(((2-amino-1,3-thiazol-4-yl)(hydroxyimino)acetyl)amino)-3-ethenyl-8-oxo-5-thia-1-azabicyclo[4.2.0]oct-2-ene-2-carboxylate (cefdinir, calcium salt, syn-isomer) pentahydrate

Excess EXAMPLE 2 and calcium acetate in 1:1 molar acetone/water at 25° C. was slurried for 1 week, filtered and air dried. HPLC analysis was performed using an Agilent 1100 series liquid chromatograph equipped with a diode array detector, degasser, quaternary pump and autosampler. The chromatographic column was a 3.9×150 mm NovaPak C18 column with 4 μm packing (Waters). The column temperature was set to 30° C., the detector wavelength was 254 nm and the reference wavelength was 360 nm. The injection volume was 20 μL. The mobile phase was a mixture of HPLC grade tetrahydrofuran, methanol and 33 mM citrate buffer (pH 2) in a 2%, 10% and 88% ratio by volume, respectively. The flow rate used was 1 mL/min. The mobile phase was filtered and degassed through a 0.22-μm nylon filter under vacuum prior to use. Product having 97% purity was obtained. Elemental analysis showed 2:1 cefdinir/calcium stoichiometry. Karl Fischer analysis showed a 10% water content, corresponding to five moles of water.

Cefdinir, calcium salt pentahydrate is stable at temperatures between about 25° C. and about 55° C. Its solubility in dimethylsulfoxide is greater than 96 mg/mL, in water, about 3 mg/mL and in acetone, acetonitrile, methanol, ethanol and dichloromethane, less than 1 mg/mL. Moisture Sorption/Desorption for crystalline cefdinir, calcium salt is shown in TABLE 1.

TABLE 1

| | | Temp | |
|---|---|---|---|
| Time (min) | Weight (mg) | (° C.) | Rel. Humid. |
| 0.1 | 6.2888 | 24.9 | 51.63 |
| 32.6 | 6.2448 | 25.0 | 4.83 |
| 48.6 | 6.2605 | 25.0 | 14.82 |
| 61.7 | 6.2704 | 25.0 | 24.85 |
| 74.6 | 6.2782 | 25.0 | 34.82 |
| 87.6 | 6.2849 | 24.9 | 44.75 |
| 100.6 | 6.2910 | 25.0 | 54.93 |
| 113.6 | 6.2957 | 25.0 | 64.86 |
| 125.2 | 6.3005 | 25.0 | 74.81 |
| 138.9 | 6.3064 | 25.0 | 84.80 |
| 220.7 | 6.3638 | 25.1 | 94.58 |
| 241.5 | 6.3265 | 25.1 | 85.41 |
| 256.2 | 6.3087 | 25.1 | 75.18 |
| 271.2 | 6.3015 | 25.1 | 65.07 |
| 284.1 | 6.2965 | 25.1 | 55.11 |
| 300.4 | 6.2899 | 25.1 | 45.16 |
| 313.1 | 6.2827 | 25.1 | 35.08 |
| 326.1 | 6.2745 | 25.0 | 25.12 |
| 338.7 | 6.2650 | 25.0 | 15.13 |
| 360.9 | 6.2484 | 25.0 | 5.03 |

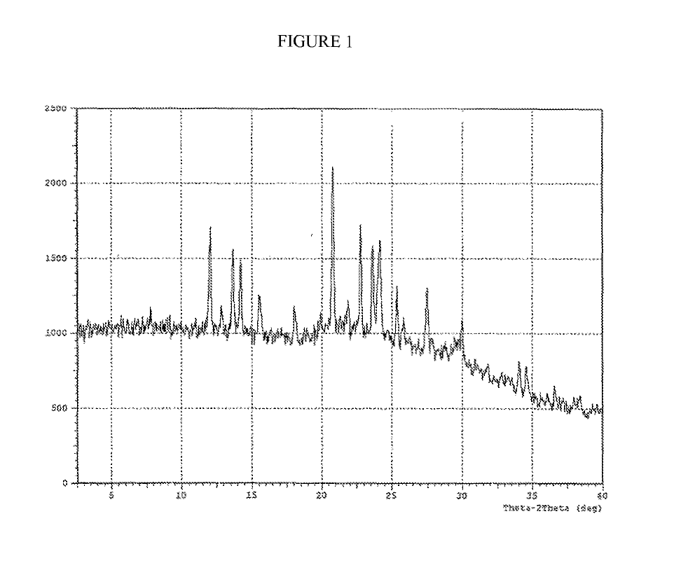

Powder X-ray powder diffraction (PXRD) analysis was performed using an Inel XRG-3000 diffractometer equipped with a CPS (Curved Position Sensitive) detector with a 2θ range of 120°. Real-time data were collected using Cu—Kα radiation starting at approximately 4° 2θ at a resolution of 0.03° 2θ. The tube voltage and amperage were set to 40 kV and 30 mA, respectively. The pattern is displayed from 2.5 to 40° 2θ. The sample was prepared for analysis by packing it into thin-walled glass capillary. Each capillary was mounted onto a goniometer head which was motorized to permit spinning of the capillary during data acquisition. The samples were analyzed for 5 minutes. Instrument calibration was performed using a silicon reference standard. The result is shown in FIG. 1.

It is meant to be understood that relative intensities of peak heights in a PXRD pattern may vary and will be dependent on variables such as the temperature, crystal size, crystal morphology or sample preparation.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions which differ from those measured with Cu—Kα radiation, which has a wavelength of 1.5478 Å.

The term “about” preceding a series of peak positions is meant to include all of the peak positions of the group which it precedes.

The term “about” preceding a series of peak positions means that all of the peaks of the group which it precedes are reported in terms of angular positions (two theta) with an allowable variability of ±0.1° as specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position. For example, if a peak from one pattern is determined to have a position of 5.2°, for comparison purposes the allowable variability allows the peak to be assigned a position in the range of 5.10°-5.3°. If a peak from another diffraction pattern has a peak position of 5.3°, for comparison purposes, the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position.

Accordingly, for example, the term “about 5.4°, 10.7°, 14.2°, 15.2°, 21.4°, 29.2° and 30.6°,” as used herein, means about 5.4°, about 10.7°, about 14.2°, about 15.2°, about 21.4°, about 29.2° and about 30.6° which, in turn, means 5.4°±0.1°, 10.7°±0.1°, 14.2°±0.1°, 15.2°+0.1°, 21.4°±0.1°, 29.2°±0.1° and 30.6°±0.1°.

A hydrated cefdinir, calcium salt may also be used to prepare Cefdinir Crystalline Form A. A solution of a hydrate of cefdinir, calcium salt, water and a solvent such as methanol, ethanol or a combination thereof between about 75° C. to about 100° C., may be acidified to about pH 1 to 4 with an acid such as hydrochloric acid and the like.

At any point during a process of this invention, an intermediate or product may by purified by means including, but not limited to, recrystallization, reverse-phase high performance liquid chromatography, ion exchange column chromatography, trituration, extraction, filtration, elution through a column of activated charcoal, nonionic adsorption resin, alumina or acidic aluminum oxide or a combination thereof.

The foregoing is meant to be illustrative of the invention and not intended to limit it to the disclosed embodiments. Variations and changes obvious to one skilled in the art are intended to be within the scope and nature of the invention as defined in the claims.

We claim:

1. An isolated crystalline hydrate of cefdinir, calcium salt.

2. Crystalline cefdinir, calcium salt pentahydrate.

3. Crystalline cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

4. A composition comprising or made with an excipient and cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

5. A method of treating bacterial infection in a mammal comprising administering thereto a therapeutically effective amount of a crystalline cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°.

6. A process for making a crystalline cefdinir, calcium salt pentahydrate, said process comprising:
providing a mixture comprising cefdinir, water and acetone;
causing crystalline cefdinir, calcium salt pentahydrate to exist in said mixture, said crystalline cefdinir, calcium salt pentahydrate, when measured with radiation at 1.54178 Å, comprising a powder diffraction pattern with at least five peaks having respective 2θ values of about 12.1°, 12.8°, 13.6°, 14.2°, 15.6°, 18.1°, 20.8°, 22.7°, 23.7°, 24.2°, 25.8°, 27.5°, 30.0°, 34.1° and 36.6°; and
isolating said crystalline cefdinir, calcium salt pentahydrate.

7. Crystalline cefdinir, calcium salt pentahydrate prepared by the method of claim 6.

8. A process for making Cefdinir Crystalline Form A, said process comprising:
providing a mixture comprising a hydrate of cefdinir, calcium salt, water and an alcohol;
causing crystalline Cefdinir Crystalline Form A to exist in said mixture, said crystalline Cefdinir Crystalline Form A, when measured with radiation at 1.54178 Å, comprising a powder diffraction pattern with at least five peaks having respective 2θ values of about 11.8°, 12.6°, 14.7°, 16.6°, 17.8°, 18.9°, 19.2°, 21.5°, 22.0°, 23.4°, 24.5°, 25.4°, 26.9°, 27.7°, 28.1° and 29.7°; and
isolating said Cefdinir Crystalline Form A.

9. The process of claim 8 wherein the hydrate of cefdinir, calcium salt is cefdinir, calcium salt pentahydrate.

10. Cefdinir Crystalline Form A prepared by the process of claim 9.

11. A process for making Cefdinir Crystalline Form A, said process comprising:
providing a mixture comprising a hydrate of cefdinir, calcium salt, water and methanol between about 75° C. to about 100° C.;
adjusting the pH of the mixture to about 1 to about 4 and causing crystalline Cefdinir Crystalline Form A to form in said mixture, said crystalline Cefdinir Crystalline Form A, when measured with radiation at 1.54178 Å, comprising a powder diffraction pattern with at least five peaks having respective 2θ values of about 11.8°, 12.6°, 14.7°, 16.6°, 17.8°, 18.9°, 19.2°, 21.5°, 22.0°, 23.4°, 24.5°, 25.4°, 26.9°, 27.7°, 28.1° and 29.7°; and
isolating said Cefdinir Crystalline Form A.

12. The process of claim 11 wherein the hydrate of cefdinir, calcium salt is cefdinir, calcium salt pentahydrate.

13. Cefdinir Crystalline Form A prepared by the process of claim 12.
